(19) United States
(12) Patent Application Publication
(10) Pub. No.: US 2023/0034846 A1
(43) Pub. Date: Feb. 2, 2023

(54) TRANSFER TAPE ARTICLES FOR PREPARING DRY ELECTRODES

(71) Applicant: 3M INNOVATIVE PROPERTIES COMPANY, St. Paul, MN (US)

(72) Inventors: Bret W. Ludwig, Oakdale, MN (US); Matthew J. Bongers, Minneapolis, MN (US)

(21) Appl. No.: 17/783,522

(22) PCT Filed: Dec. 10, 2020
(86) PCT No.: PCT/IB2020/061790

§ 371 Date: Jun. 8, 2022

Related U.S. Application Data
(60) Provisional application No. 62/949,123, filed on Dec. 17, 2019.

(51) Int. Cl. C09J 9/02; C09J 11/04; C09J 7/38; A61B 5/259

(52) U.S. Cl. CPC C09J 9/02; C09J 11/04; C09J 7/385; A61B 5/259; A61B 2562/125

(57) ABSTRACT

Transfer tape articles are suitable for preparing dry electrodes. The transfer tape articles include a release liner and a conductive transfer tape layer adjacent to the release liner. The conductive transfer tape layer includes a layer of adhesive and a discontinuous layer of electrically conductive shaped particles, where the shaped particles have at least one point. The adhesive envelopes the conductive particles, and at least one point of the electrically conductive particles protrudes from the conductive transfer tape layer. The conductive transfer tape layer can be a single layer of adhesive or a multi-layer construction including a first adhesive layer, a support layer, and a second adhesive layer.

### FIELD OF THE DISCLOSURE

Disclosed herein are transfer tape articles that are suitable for preparing dry electrodes.

### BACKGROUND

Electrodes for measuring biopotential are used extensively in modern clinical and biomedical applications. The applications encompass numerous physiological tests including electrocardiography (ECG), electroencephalography (EEG), electrical impedance tomography (EIT), electromyography (EMG), and electro-oculography (EOG). The electrodes for these types of physiological tests function as a transducer by transforming the electric potential or biopotentials within the body into an electric voltage that can be measured by conventional measurement and recording devices.

### SUMMARY

Disclosed herein are transfer tape articles that are suitable for preparing dry electrodes, dry electrodes, and methods for preparing dry electrodes. Disclosed herein are transfer tape articles. In some embodiments, the transfer tape articles comprise a release liner with a first major surface and a second major surface and a conductive transfer tape layer with a first major surface and a second major surface, where the first major surface of the conductive transfer tape layer is adjacent to the second major surface of the release liner. The conductive transfer tape layer comprises at least a first layer of adhesive comprising a first major surface and a second major surface, where a portion of first major surface of the first adhesive layer is in contact with the second major surface of the release liner, and a discontinuous layer of electrically conductive particles. At least some of the electrically conductive particles are in contact with the second major surface of the release liner. The electrically conductive particles comprise shaped particles with at least one point. The first layer of adhesive envelopes the conductive particles, and at least one point of at least one of the electrically conductive particles protrudes from the second major surface of the conductive transfer tape layer. In some embodiments, the conductive transfer tape layer instead of being a single layer of adhesive comprises a multi-layer construction comprising a first adhesive layer, a support layer, and a second adhesive layer.

Also disclosed herein are electrodes. In some embodiments, the electrodes comprise a substrate with a conductive surface and a conductive transfer tape layer in contact with at least a portion of the conductive surface of the substrate. The conductive transfer tape layers are described above. In some embodiments, the substrate comprises an electronic device.

Also disclosed are methods of preparing electrodes. In some embodiments, the method comprises providing a substrate with a conductive surface, providing a conductive transfer tape article with a first major surface and a second major surface with a release liner in contact with the first major surface of the conductive transfer tape layer as described above, removing the release liner from the conductive transfer tape article to expose the first major surface of the conductive transfer tape layer, and contacting first major surface of the conductive transfer tape article to the conductive surface of the substrate.

### BRIEF DESCRIPTION OF THE DRAWINGS

The present application may be more completely understood in consideration of the following detailed description of various embodiments of the disclosure in connection with the accompanying drawings.

FIG. 1 is a cross sectional view of an embodiment of a conductive transfer tape article of the present disclosure.

FIG. 2 is a cross sectional view of an embodiment of another conductive transfer tape article of the present disclosure.

FIG. 3 is cross sectional view of an embodiment of a dry electrode of this the present disclosure.

In the following description of the illustrated embodiments, reference is made to the accompanying drawings, in which is shown by way of illustration, various embodiments in which the disclosure may be practiced. It is to be understood that the embodiments may be utilized and structural changes may be made without departing from the scope of the present disclosure. The figures are not necessarily to scale. Like numbers used in the figures refer to like components. However, it will be understood that the use of a number to refer to a component in a given figure is not intended to limit the component in another figure labeled with the same number.

### DETAILED DESCRIPTION

Dual-sided tapes, also called “transfer tapes” are adhesive tapes that have adhesive on both exposed surfaces. In some transfer tapes, the exposed surfaces are simply the two surfaces of a single adhesive layer. Other transfer tapes are multi-layer transfer tapes with at least two adhesive layers that may be the same or different, and in some instances intervening layers that may not be adhesive layers. Disclosed herein are transfer tape articles that can be used to form electrodes. These electrodes are “dry” electrodes as are described in greater detail below.

Electrodes for measuring biopotential are used extensively in modern clinical and biomedical applications. The applications encompass numerous physiological tests including electrocardiography (ECG), electroencephalography (EEG), electrical impedance tomography (EIT), electromyography (EMG), and electro-oculography (EOG).

The electrodes for these types of physiological tests function as a transducer by transforming the electric potential or biopotentials within the body into an electric voltage that can be measured by conventional measurement and recording devices.

In general, such electrodes are attached to the surface of the skin. A difficulty with electrodes placed on the surface of the skin is that the outer layer of skin, the stratum corneum, lacks moisture, and this lack of moisture gives high impedance. This high impedance results from the lack of ion mobility due to the lack of moisture in the stratum corneum.

Electrical conduction in the body is based on the movement of ions rather than the movement of electrons as found in metallic conductors. In order to register an electronic signal from the body an electrode must transform ionic conduction to electronic conduction. The vast majority of electrodes available today achieve this transduction through the use of silver/silver chloride reduction/oxidation reactions. A hydrogel containing Ag⁺and Cl⁻ions adjacent to the skin wets the stratum corneum and enables ion mobility between the skin and the electrode. Transduction occurs within the electrode at the interface of the hydrogel and an electronically conducting material (typically a metal snap or layer of conductive carbon composite). The conducting material is coated with silver, enabling the following reversible reactions and thus the detection of electrical pulses within the body.

Ag⁺+e⁻ [symbol] Ag(s)

AgCl(s)+e⁻ [symbol] Ag(s)+Cl⁻

One difficulty with the use of hydrogel-containing electrodes is that the water in the hydrogel is subject to evaporation. Therefore, when in use, the hydrogel can lose water and become ineffective. Additionally, the loss of water from the hydrogel as the electrode is stored and transported is a significant challenge. In order to prevent water loss from the hydrogel prior to its use, often expensive packaging, such as foil-lined envelopes, is used to increase the limited shelf life of such electrodes.

Therefore, considerable effort has been expended to the development of “dry” electrodes that do not utilize hydrogels. To make dry electrodes, many attempts have focused on an alternative to using a hydrogel to hydrate the dry stratum corneum, which is to use small structures to penetrate the stratum corneum and access the more moisture-rich layers of skin that lie beneath. Generally, the structures are coated with materials that will form a reduction/oxidation couple (typically silver and silver chloride) when in the presence of moisture. Examples of such small structures that have been used include microneedles or similar small, pointed structures to penetrate the stratum corneum to deeper layers of the skin with more moisture where ions are more mobile, enabling conduction. In U.S. Provisional Patent Application No. U.S. 62/760,351 dry electrodes are described that include microreplicated particles in an adhesive matrix.

The methods of manufacturing these electrodes, typically involve injection molding or other similar processes with subsequent coating of the formed microneedle structures with the redox couple are batch processes which are relatively high cost processes. These manufacturing issues have limited dry electrodes to niche markets. Therefore, new processes for preparing dry electrodes in an economical way to permit these electrodes to compete in the marketplace with the relatively easily produced wet electrodes are needed.

In this disclosure, methods of producing dry electrodes are described which involve the use of transfer tape articles. The transfer tape articles comprise microreplicated particles that can be prepared in a continuous process. Such particles have been prepared for use in abrasive articles and can be utilized to form skin-penetrating structures in an electrode construction. Further, these particles can be coated with the redox materials in bulk processes. These coated particles are then incorporated into transfer tape articles.

Unless otherwise indicated, all numbers expressing feature sizes, amounts, and physical properties used in the specification and claims are to be understood as being modified in all instances by the term “about.” Accordingly, unless indicated to the contrary, the numerical parameters set forth in the foregoing specification and attached claims are approximations that can vary depending upon the desired properties sought to be obtained by those skilled in the art utilizing the teachings disclosed herein. The recitation of numerical ranges by endpoints includes all numbers subsumed within that range (e.g. 1 to 5 includes 1, 1.5, 2, 2.75, 3, 3.80, 4, and 5) and any range within that range.

As used in this specification and the appended claims, the singular forms “a”, “an”, and “the” encompass embodiments having plural referents, unless the content clearly dictates otherwise. For example, reference to “a layer” encompasses embodiments having one, two or more layers. As used in this specification and the appended claims, the term “or” is generally employed in its sense including “and/or” unless the content clearly dictates otherwise.

The term “adhesive” as used herein refers to polymeric compositions useful to adhere together two adherends. Examples of adhesives are pressure sensitive adhesives and gel adhesives.

Pressure sensitive adhesive compositions are well known to those of ordinary skill in the art to possess properties including the following: (1) aggressive and permanent tack, (2) adherence with no more than finger pressure, (3) sufficient ability to hold onto an adherend, and (4) sufficient cohesive strength to be cleanly removable from the adherend. Materials that have been found to function well as pressure sensitive adhesives are polymers designed and formulated to exhibit the requisite viscoelastic properties resulting in a desired balance of tack, peel adhesion, and shear holding power. Obtaining the proper balance of properties is not a simple process.

As used herein, the term “gel adhesive” refers to a tacky semi-solid crosslinked matrix containing a liquid or a fluid that is capable of adhering to one or more substrates.

The gel adhesives may have some properties in common with pressure sensitive adhesives, but they are not pressure sensitive adhesives. “Hydrogel adhesives” are gel adhesives that have water as the fluid contained within the crosslinked matrix.

The term “(meth)acrylate” refers to monomeric acrylic or methacrylic esters of alcohols. Acrylate and methacrylate monomers or oligomers are referred to collectively herein as “(meth)acrylates”. Materials referred to as “(meth)acrylate functional” are materials that contain one or more (meth)acrylate groups.

As used herein, the terms “shaped abrasive particle” and “shaped particle” are used interchangeably and refer to ceramic particles that can be used as abrasive particles with at least a portion of the particle having a predetermined shape that is replicated from a mold cavity used to form the shaped precursor particle. The shaped particle will generally have a predetermined geometric shape that substantially replicates the mold cavity that was used to form the shaped particle. Shaped particles as used herein excludes abrasive particles obtained by a mechanical crushing operation.

The terms “room temperature” and “ambient temperature” are used interchangeably to mean temperatures in the range of 20° C. to 25° C.

The terms “Tg” and “glass transition temperature” are used interchangeably. If measured, Tg values are determined by Differential Scanning calorimetry (DSC) at a scan rate of 10° C./minute, unless otherwise indicated. Typically, Tg values for copolymers are not measured but are calculated using the well-known Fox Equation, using the monomer Tg values provided by the monomer supplier, as is understood by one of skill in the art.

Disclosed herein are transfer tape articles. In some embodiments, the transfer tape articles comprise a release liner with a first major surface and a second major surface, and a conductive transfer tape layer with a first major surface and a second major surface, where the conductive transfer tape layer is adjacent to the second major surface of the release liner. The conductive transfer tape layer comprises at least a first layer of adhesive comprising a first major surface and a second major surface, wherein a portion of the first major surface of the first adhesive layer is in contact with the second major surface of the release liner, and a discontinuous layer of electrically conductive particles wherein at least some of the electrically conductive particles are in contact with the second major surface of the release liner. The electrically conductive particles comprise shaped particles with at least one point and where the first layer of adhesive envelopes the conductive particles, and at least one point of at least one of the electrically conductive particles protrudes from the second major surface of the conductive transfer tape layer. Thus, an electrical pathway thorough the conductive transfer tape layer via the electrically conductive particles is present.

The transfer tape article comprises a release liner. A wide variety of release liners are suitable. Release liners are well understood in the adhesive arts as film articles from which an adhesive can be readily removed. Exemplary release liners include those prepared from paper (e.g., Kraft paper) or polymeric material (e.g., polyolefins such as polyethylene or polypropylene, ethylene vinyl acetate, polyurethanes, polyesters such as polyethylene terephthalate, and the like, and combinations thereof). At least some release liners are coated with a layer of a release agent such as a silicone-containing material or a fluorocarbon-containing material. Exemplary release liners include, but are not limited to, liners commercially available from CP Film (Martinsville, Va.) under the trade designation “T-30” and “T-10” that have a silicone release coating on polyethylene terephthalate film.

The transfer tape article also comprises a conductive transfer tape layer with a first major surface and a second major surface, where the conductive transfer tape layer is adjacent to the second major surface of the release liner. The conductive transfer tape layer comprises a first layer of adhesive comprising a first major surface and a second major surface and a discontinuous layer of electrically conductive particles. A portion of the first major surface of the first adhesive layer, and at least some of the electrically conductive particles are in contact with the second major surface of the release liner. The electrically conductive particles comprise shaped particles with at least one point. The first layer of adhesive envelopes the conductive particles, and at least one point of at least one of the electrically conductive particles protrudes from the second major surface of the conductive transfer tape layer.

The conductive transfer tape layer can have a wide range of thicknesses. Typically, the conductive transfer tape layer has a thickness that is 25-250 micrometers less than the at least one dimension of the shaped particles.

The transfer tape articles may also further comprise a second release liner with a first major surface and a second major surface, where the first major surface of the second release liner is in contact with the second major surface of the conductive transfer tape layer. In this way, the first major surface of the second release liner is in contact with a portion of second major surface of the first adhesive layer and also is in contact with a point of at least one conductive particle.

A wide range of adhesives are suitable for use as the first layer of adhesive in the conductive transfer tape layer. In some embodiments, the first layer of adhesive comprises a layer of pressure sensitive adhesive. Pressure sensitive adhesives are very suitable for use in the conductive transfer tape layer, as they can function to hold the electrically conductive particles in place and also to adhere the transfer tape article to the skin of a user, or to another device or article. That is one of the advantages of the conductive transfer tape layers of the present disclosure, since they have two exposed adhesive surfaces, one adhesive surface can be used to adhere the transfer tape layer to a device and the other adhesive surface can be used to adhesive the transfer tape layer to human skin. In this way the transfer tape can form a dry electrode.

A wide range of pressure sensitive adhesives are suitable for use as the supporting layer. Useful pressure sensitive adhesives include those based on natural rubbers, synthetic rubbers, styrene block copolymers, polyvinyl ethers, acrylics, poly-α-olefins, silicones, polyurethanes or polyureas.

Useful natural rubber pressure sensitive adhesives generally contain masticated natural rubber, from 25 parts to 300 parts of one or more tackifying resins to 100 parts of natural rubber, and typically from 0.5 to 2.0 parts of one or more antioxidants. Natural rubber may range in grade from a light pale crepe grade to a darker ribbed smoked sheet and includes such examples as CV-60, a controlled viscosity rubber grade and SMR-5, a ribbed smoked sheet rubber grade.

Tackifying resins used with natural rubbers generally include, but are not limited to, wood rosin and its hydrogenated derivatives; terpene resins of various softening points, and petroleum-based resins, such as, the “ESCOREZ 1300” series of C5 aliphatic olefin-derived resins from Exxon, and “PICCOLYTE S” series, polyterpenes from Hercules, Inc. Antioxidants are used to retard the oxidative attack on natural rubber, which can result in loss of the cohesive strength of the natural rubber adhesive. Useful antioxidants include, but are not limited to, amines, such as N-N′-di-B-naphthyl-1,4-phenylenediamine, available as “AGERITE D”; phenolics, such as 2,5-di-(t-amyl) hydroquinone, available as “SANTOVAR A”, available from Monsanto Chemical Co., tetrakis[methylene 3-(3′,5′-di-tert-butyl-4′-hydroxyphenyl)propionate]methane, available as “IRGANOX 1010” from Ciba-Geigy Corp., and 2-2′-methylenebis(4-methyl-6-tert butyl phenol), available as Antioxidant 2246; and dithiocarbamates, such as zinc dithiodibutyl carbamate. Other materials can be added to natural rubber adhesives for special purposes, wherein the additives can include plasticizers, pigments, and curing agents to partially vulcanize the pressure sensitive adhesive.

Another useful class of pressure sensitive adhesives are those comprising synthetic rubber. Such adhesives are generally rubbery elastomers, which are either self-tacky or non-tacky and require tackifiers.

Self-tacky synthetic rubber pressure sensitive adhesives include for example, butyl rubber, a copolymer of isobutylene with less than 3 percent isoprene, polyisobutylene, a homopolymer of isoprene, polybutadiene, such as “TAKTENE 220 BAYER” or styrene/butadiene rubber. Butyl rubber pressure sensitive adhesives often contain an antioxidant such as zinc dibutyldithiocarbamate. Polyisobutylene pressure sensitive adhesives do not usually contain antioxidants. Synthetic rubber pressure sensitive adhesives, which generally require tackifiers, are also generally easier to melt process. They comprise polybutadiene or styrene/butadiene rubber, from 10 parts to 200 parts of a tackifier, and generally from 0.5 to 2.0 parts per 100 parts rubber of an antioxidant such as “IRGANOX 1010”. An example of a synthetic rubber is “AMERIPOL 1011A”, a styrene/butadiene rubber available from BF Goodrich. Tackifiers that are useful include derivatives of rosins such as “FORAL 85”, a stabilized rosin ester from Hercules, Inc., the “SNOWTACK” series of gum rosins from Tenneco, and the “AQUATAC” series of tall oil rosins from Sylvachem; and synthetic hydrocarbon resins such as the “PICCOLYTE A” series, polyterpenes from Hercules, Inc., the “ESCOREZ 1300” series of C5 aliphatic olefin-derived resins, the “ESCOREZ 2000” Series of C9 aromatic/aliphatic olefin-derived resins, and polyaromatic C₉ resins, such as the “PICCO 5000” series of aromatic hydrocarbon resins, from Hercules, Inc. Other materials can be added for special purposes, including hydrogenated butyl rubber, pigments, plasticizers, liquid rubbers, such as “VISTANEX LMMH” polyisobutylene liquid rubber available from Exxon, and curing agents to vulcanize the adhesive partially.

Styrene block copolymer pressure sensitive adhesives generally comprise elastomers of the A-B or A-B-A type, where A represents a thermoplastic polystyrene block and B represents a rubbery block of polyisoprene, polybutadiene, or poly(ethylene/butylene), and resins. Examples of the various block copolymers useful in block copolymer pressure sensitive adhesives include linear, radial, star and tapered styrene-isoprene block copolymers such as “KRATON D1107P”, available from Shell Chemical Co., and “EUROPRENE SOL TE 9110”, available from EniChem Elastomers Americas, Inc.; linear styrene-(ethylene-butylene) block copolymers such as “KRATON G1657”, available from Shell Chemical Co.; linear styrene-(ethylene-propylene) block copolymers such as “KRATON G1750X”, available from Shell Chemical Co.; and linear, radial, and star styrene-butadiene block copolymers such as “KRATON D1118X”, available from Shell Chemical Co., and “EUROPRENE SOL TE 6205”, available from EniChem Elastomers Americas, Inc. The polystyrene blocks tend to form domains in the shape of spheroids, cylinders, or plates that causes the block copolymer pressure sensitive adhesives to have two-phase structures. Resins that associate with the rubber phase generally develop tack in the pressure sensitive adhesive. Examples of rubber phase associating resins include aliphatic olefin-derived resins, such as the “ESCOREZ 1300” series and the “WINGTACK” series, available from Goodyear; rosin esters, such as the “FORAL” series and the “STAYBELITE” Ester 10, both available from Hercules, Inc.; hydrogenated hydrocarbons, such as the “ESCOREZ 5000” series, available from Exxon; polyterpenes, such as the “PICCOLYTE A” series; and terpene phenolic resins derived from petroleum or terpentine sources, such as “PICCOFYN A100”, available from Hercules, Inc. Resins that associate with the thermoplastic phase tend to stiffen the pressure sensitive adhesive. Thermoplastic phase associating resins include polyaromatics, such as the “PICCO 6000” series of aromatic hydrocarbon resins, available from Hercules, Inc.; coumarone-indene resins, such as the “CUMAR” series, available from Neville; and other high-solubility parameter resins derived from coal tar or petroleum and having softening points above about 85° C., such as the “AMOCO 18” series of alpha-methyl styrene resins, available from Amoco, “PICCOVAR 130” alkyl aromatic polyindene resin, available from Hercules, Inc., and the “PICCOTEX” series of alpha-methyl styrene/vinyltoluene resins, available from Hercules. Other materials can be added for special purposes, including rubber phase plasticizing hydrocarbon oils, such as, “TUFFLO 6056”, available from Lyondell Petrochemical Co., Polybutene-8 from Chevron, “KAYDOL”, available from Witco, and “SHELLFLEX 371”, available from Shell Chemical Co.; pigments; antioxidants, such as “IRGANOX 1010” and “IRGANOX 1076”, both available from Ciba-Geigy Corp., “BUTAZATE”, available from Uniroyal Chemical Co., “CYANOX LDTP”, available from American Cyanamid, and “BUTASAN”, available from Monsanto Co.; antiozonants, such as “NBC”, a nickel dibutyldithiocarbamate, available from DuPont; liquid rubbers such as “VISTANEX LMMH” polyisobutylene rubber; and ultraviolet light inhibitors, such as “IRGANOX 1010” and “TINUVIN P”, available from Ciba-Geigy Corp.

Polyvinyl ether pressure sensitive adhesives are generally blends of homopolymers of vinyl methyl ether, vinyl ethyl ether or vinyl iso-butyl ether, or blends of homopolymers of vinyl ethers and copolymers of vinyl ethers and acrylates to achieve desired pressure sensitive properties. Depending on the degree of polymerization, homopolymers may be viscous oils, tacky soft resins or rubber-like substances. Polyvinyl ethers used as raw materials in polyvinyl ether adhesives include polymers based on: vinyl methyl ether such as “LUTANOL M 40”, available from BASF, and “GANTREZ M 574” and “GANTREZ 555”, available from ISP Technologies, Inc.; vinyl ethyl ether such as “LUTANOL A 25”, “LUTANOL A 50” and “LUTANOL A 100”; vinyl isobutyl ether such as “LUTANOL 130”, “LUTANOL 160”, “LUTANOL IC”, “LUTANOL I60D” and “LUTANOL I 65D”; methacrylate/vinyl isobutyl ether/acrylic acid such as “ACRONAL 550 D”, available from BASF. Antioxidants useful to stabilize the polyvinylether pressure sensitive adhesive include, for example, “IONOX 30” available from Shell, “IRGANOX 1010” available from Ciba-Geigy, and antioxidant “ZKF” available from Bayer Leverkusen. Other materials can be added for special purposes as described in BASF literature including tackifiers, plasticizers and pigments.

Acrylic pressure sensitive adhesives generally have a glass transition temperature of about −20° C. or less and may comprise from 100 to 80 weight percent of a C₃-C₁₂ alkyl ester component such as, for example, isooctyl acrylate, 2-ethylhexyl acrylate and n-butyl acrylate and from 0 to 20 weight percent of a polar component such as, for example, acrylic acid, methacrylic acid, ethylene-vinyl acetate units, N-vinylpyrrolidone, and styrene macromer. Generally, the acrylic pressure sensitive adhesives comprise from 0 to 20 weight percent of acrylic acid and from 100 to 80 weight percent of isooctyl acrylate.

The acrylic pressure sensitive adhesives may be self-tacky or tackified. Useful tackifiers for acrylics are rosin esters such as “FORAL 85”, available from Hercules, Inc., aromatic resins such as “PICCOTEX LC-55WK”, aliphatic resins such as “PICCOTAC 95”, available from Hercules, Inc., and terpene resins such as a-pinene and B-pinene, available as “PICCOLYTE A-115” and “ZONAREZ B-100” from Arizona Chemical Co. Other materials can be added for special purposes, including hydrogenated butyl rubber, pigments, and curing agents to vulcanize the adhesive partially.

Poly-α-olefin pressure sensitive adhesives, also called a poly(l-alkene) pressure sensitive adhesives, generally comprise either a substantially uncrosslinked polymer or an uncrosslinked polymer that may have radiation activatable functional groups grafted thereon as described in U.S. Pat. No. 5,209,971 (Babu, et al). The poly-α-olefin polymer may be self tacky and/or include one or more tackifying materials. If uncrosslinked, the inherent viscosity of the polymer is generally between about 0.7 and 5.0 dL/g as measured by ASTM D 2857-93, “Standard Practice for Dilute Solution Viscosity of Polymers”. In addition, the polymer generally is predominantly amorphous. Useful poly-α-olefin polymers include, for example, C₃-C₁₈ poly(l-alkene) polymers, generally C₅-C₁₂α-olefins and copolymers of those with C₃ or C₆-C₈ and copolymers of those with C₃.

Tackifying materials are typically resins that are miscible in the poly-α-olefin polymer. The total amount of tackifying resin in the poly-α-olefin polymer ranges from 0 to 150 parts by weight per 100 parts of the poly-α-olefin polymer depending on the specific application. Useful tackifying resins include resins derived by polymerization of C₅ to C₉ unsaturated hydrocarbon monomers, polyterpenes, synthetic polyterpenes and the like.

Examples of such commercially available resins based on a C₅ olefin fraction of this type are “WINGTACK 95” and “WINGTACK 15” tackifying resins available from Goodyear Tire and Rubber Co. Other hydrocarbon resins include “REGALREZ 1078” and “REGALREZ 1126” available from Hercules Chemical Co., and “ARKON P115” available from Arakawa Chemical Co. Other materials can be added for special purposes, including antioxidants, fillers, pigments, and radiation activated crosslinking agents.

Silicone pressure sensitive adhesives comprise two major components, a polymer or gum, and a tackifying resin. The polymer is typically a high molecular weight polydimethylsiloxane or polydimethyldiphenylsiloxane, that contains residual silanol functionality (SiOH) on the ends of the polymer chain, or a block copolymer comprising polydiorganosiloxane soft segments and urea or oxamide terminated hard segments. The tackifying resin is generally a three-dimensional silicate structure that is endcapped with trimethylsiloxy groups (OSiMe₃) and also contains some residual silanol functionality. Examples of tackifying resins include SR 545, from General Electric Co., Silicone Resins Division, Waterford, N.Y., and MQD-32-2 from Shin-Etsu Silicones of America, Inc., Torrance, Calif Manufacture of typical silicone pressure sensitive adhesives is described in U.S. Pat. No. 2,736,721 (Dexter). Manufacture of silicone urea block copolymer pressure sensitive adhesive is described in U.S. Pat. No. 5,214,119 (Leir, et al). Other materials can be added for special purposes, including pigments, plasticizers, and fillers. Fillers are typically used in amounts from 0 parts to 10 parts per 100 parts of silicone pressure sensitive adhesive. Examples of fillers that can be used include zinc oxide, silica, carbon black, pigments, metal powders and calcium carbonate. One particularly suitable class or siloxane-containing pressure sensitive adhesives are those with oxamide terminated hard segments such as those described in U.S. Pat. No. 7,981,995 (Hays) and U.S. Pat. No. 7,371,464 (Sherman).

Polyurethane and polyurea pressure sensitive adhesives useful in this disclosure include, for example, those disclosed in WO 00/75210 (Kinning et al.) and in U.S. Pat. No. 3,718,712 (Tushaus); U.S. Pat. No. 3,437,622 (Dahl); and 5,591,820 (Kydonieus et al.).

Particularly useful pressure sensitive adhesives are based on at least one poly(meth)acrylate (i.e., a (meth)acrylic pressure sensitive adhesive). Particularly desirable poly(meth)acrylates are derived from: (A) at least one monoethylenically unsaturated alkyl (meth) acrylate monomer (i.e., alkyl acrylate and alkyl methacrylate monomer); and (B) at least one monoethylenically unsaturated free-radically copolymerizable reinforcing monomer. The reinforcing monomer has a homopolymer glass transition temperature (Tg) higher than that of the alkyl (meth)acrylate monomer and is one that increases the glass transition temperature and cohesive strength of the resultant copolymer.

Generally, the monomers used in preparing the pressure sensitive adhesive include: (A) a monoethylenically unsaturated alkyl (meth)acrylate monomer that, when homopolymerized, generally has a glass transition temperature (Tg) of no greater than about 0° C.; and (B) a monoethylenically unsaturated free-radically copolymerizable reinforcing monomer that, when homopolymerized, generally has a glass transition temperature of at least about 10° C. The glass transition temperatures of the copolymers of monomers A and B can be measured by differential scanning calorimetry, but more typically the Tg is calculated using the well-known Fox equation utilizing the homopolymer Tg values supplied by the monomer supplier.

Monomer A, which is a monoethylenically unsaturated alkyl acrylate or methacrylate (i.e., (meth)acrylic acid ester), contributes to the flexibility and tack of the copolymer of the adhesive component of the fibers. Generally, monomer A has a homopolymer Tg of no greater than about 0° C. Typically, the alkyl group of the (meth)acrylate has an average of about 4 to about 20 carbon atoms, and more generally, an average of about 4 to about 14 carbon atoms. The alkyl group can optionally contain oxygen atoms in the chain thereby forming ethers or alkoxy ethers, for example. Examples of monomer A include, but are not limited to, 2-methylbutyl acrylate, isooctyl acrylate, lauryl acrylate, 4-methyl-2-pentyl acrylate, isoamyl acrylate, sec-butyl acrylate, n-butyl acrylate, n-hexyl acrylate, 2-ethylhexyl acrylate, n-octyl acrylate, n-decyl acrylate, isodecyl acrylate, isodecyl methacrylate, and isononyl acrylate. Other examples include, but are not limited to, poly-ethoxylated or -propoxylated methoxy (meth)acrylates such as acrylates of CARBOWAX (commercially available from Union Carbide) and NK ester AM90G (commercially available from Shin Nakamura Chemical, Ltd., Japan). Particularly suitable monoethylenically unsaturated (meth)acrylates that can be used as monomer A include isooctyl acrylate, 2-ethyl-hexyl acrylate, and n-butyl acrylate.

Monomer B, which is a monoethylenically unsaturated free-radically copolymerizable reinforcing monomer, increases the glass transition temperature and cohesive strength of the copolymer. Generally, monomer B has a homopolymer Tg of at least about 10° C. Useful examples of monomer B can be polar or non-polar. Typically, monomer B is a reinforcing (meth)acrylic monomer, including an acrylic acid, a methacrylic acid, an acrylamide, or a (meth)acrylate. Examples of monomer B include, but are not limited to, acrylamides, such as acrylamide, methacrylamide, N-methyl acrylamide, N-ethyl acrylamide, N-hydroxyethyl acrylamide, diacetone acrylamide, N,N-dimethyl acrylamide, N, N-diethyl acrylamide, N-ethyl-N-aminoethyl acrylamide, N-ethyl-N-hydroxyethyl acrylamide, N,N-dihydroxyethyl acrylamide, t-butyl acrylamide, N,N-dimethylaminoethyl acrylamide, and N-octyl acrylamide. Other examples of monomer B include itaconic acid, crotonic acid, maleic acid, fumaric acid, 2,2-(diethoxy)ethyl acrylate, 2-hydroxyethyl acrylate or methacrylate, 3-hydroxypropyl acrylate or methacrylate, methyl methacrylate, isobornyl acrylate, 2-(phenoxy)ethyl acrylate or methacrylate, biphenylyl acrylate, t-butylphenyl acrylate, cyclohexyl acrylate, dimethyladamantyl acrylate, 2-naphthyl acrylate, phenyl acrylate, N-vinyl formamide, N-vinyl acetamide, N-vinyl pyrrolidone, and N-vinyl caprolactam. Particularly suitable reinforcing acrylic monomers that can be used as monomer B include acrylic acid and acrylamide

Generally, the acrylate copolymer is formulated to have a resultant Tg of less than about 25° C. and more typically, less than about 0° C. Such acrylate copolymers typically include about 60 parts to about 98 parts per hundred of at least one monomer A and about 2 parts to about 40 parts per hundred of at least one monomer B. In some embodiments, the acrylate copolymers have about 85 parts to about 98 parts per hundred or at least one monomer A and about 2 parts to about 15 parts of at least one monomer B.

A crosslinking agent can be used if so desired to build the molecular weight and the strength of the copolymer of the adhesive. Typically, the crosslinking agent is one that is copolymerized with monomers A and B. The crosslinking agent may produce chemical crosslinks (e.g., covalent bonds or ionic bonds). Alternatively, it may produce thermal reversible physical crosslinks that result, for example, from the formation of reinforcing domains due to phase separation of hard segments (i.e., those having a Tg higher than room temperature, generally higher than 70° C.) and/or acid/base interactions (i.e., those involving functional groups within the same polymer or between polymers or between a polymer and an additive). In some embodiments, crosslinking occurs through the use of macromers, such as the styrene macromers of U.S. Pat. No. 4,554,324 (Husman), or polymeric ionic crosslinking as described in WO 99/42536. Suitable crosslinking agents Are also disclosed in U.S. Pat. No. 4,737,559 (Kellen), 5,506,279 (Babu et al.), and 6,083,856 (Joseph et al.).

The conductive transfer tape layers of this disclosure also include a discontinuous layer of electrically conductive particles. By discontinuous layer it is meant that the layer includes discrete particles that are not all in contact with each other.

A wide variety of electrically conductive particles are suitable for use in the sheet articles of this disclosure. Generally, the electrically conductive particles comprise non-compressive shaped particles with a conductive metal coating. Typically, the conductive particles are prepared from particles prepared by methods used to make shaped abrasive particles, but the conductive particles can have a wide variety of shapes including shapes such as a caltrops. The conductive metal coating may be a multi-layer coating, for example a conductive metal coating may cover the entire or essentially the entire surface of the shaped particles, and a secondary coating, such as a silver coating or a silver/silver chloride coating, may be selectively coated over all or a portion of the first conductive metal coating. In some embodiments, the electrically conductive particles comprise particles that are entirely coated with silver, and a portion of this coating is converted to a silver/silver chloride coating or a portion of the silver coating could be overcoated with a silver chloride coating. In other embodiments, the particles are entirely coated with a conductive coating and a portion of the particles are then coated with silver and converted to a silver/silver chloride coating. While a wide range of sizes and shapes are suitable for the shaped particles, at least one dimension of the shaped particles is 175-1,500 micrometers.

In some embodiments, the non-compressive shaped particles comprise ceramic shaped particles. Examples of suitable ceramic shaped particles are shaped abrasive alumina particles with a sloping sidewall as described in U.S. Pat. No. 8,142,531, or shaped abrasive zirconia particles as described in US Patent Publication No. 2016/0214903. Shaped abrasive particles are particularly desirable for use in forming the articles of this disclosure since they can be mass produced with known technologies and coated with an electrically conductive coating to form particles with at least one point. These pointed, electrically conductive particles, when formed into a dry electrode, are non-compressive and are capable of penetrating the stratum corneum. The current disclosure is not related to the preparation of non-compressive ceramic particles but rather to their use in a new and unexpected way to prepare transfer tape articles and dry electrodes.

The formed ceramic particles can be coated with metal using a variety of different techniques. Among the suitable methods are physical vapor deposition. Physical vapor deposition (PVD) describes a variety of vacuum deposition methods which can be used to produce thin films and coatings. PVD is characterized by a process in which the material goes from a condensed phase to a vapor phase and then back to a thin film condensed phase. The most common PVD processes are sputtering and evaporation.

Typically, the ceramic particles are coated with silver using the methods and apparatus described in U.S. Pat. Nos. 4,612,242 and 7,727,931, and US Patent Publication No. 2014/0363554.

In some embodiments, the electrically conductive particles comprise non-compressive shaped particles with a conductive metal coating wherein at least one dimension of the shaped particles is 175-1,500 micrometers.

The conductive transfer tape layer may be a multi-layer construction. In some embodiments, the multi-layer construction comprises a continuous or a discontinuous non-conductive support layer with a first major surface and a second major surface wherein the first major surface of the support layer is in contact with the second major surface of the first layer of adhesive, and a second adhesive layer with a first major surface and a second major surface, where the first major surface of the second adhesive layer is in contact with the second major surface of the support layer. In these embodiments, the support layer and the second adhesive layer also envelope the electrically conductive particles, and at least one point from at least one particle protrudes through the second major surface of the second adhesive layer.

In some embodiments, the multi-layer conductive transfer tape further comprise a second release liner with a first major surface and a second major surface, where the first major surface of the second release liner is in contact with at least one electrically conductive particle protruding from the second major surface of the transfer tape layer and is also in contact with at least a portion of the second major surface of the transfer tape layer.

A wide variety of conductive or non-conductive support layers are suitable. In some embodiments, the support layer is an essentially continuous layer comprising a film, a web, a sheet, or a foam. In many embodiments, the support layer comprises a layer suitable for use in a medical article. Examples of such layers are breathable conformable material layers with high moisture vapor permeability. Examples of such layers, methods of making such layers, and methods for testing their permeability are described, for example, in U.S. Pat. Nos. 3,645,835 and 4,595,001. Typically, such layers are non-conductive but can be conductive such as a non-woven web comprising conductive fibers or a film comprising a conductive layer.

Examples of particularly suitable support layers can be found in U.S. Pat. Nos. 5,088,483 and 5,160,315, and include elastomeric polyurethane, polyester, or polyether block amide films. Support layers may also be sheets. Examples of suitable sheets include, for example, paper and similar materials, and may be modified. These films have a combination of desirable properties including conformability and ease of penetration by the conductive particles.

Generally, the support layer has a thickness of from 10-500 micrometers, in some embodiments 12-50 micrometers.

A wide variety of adhesives, typically pressure sensitive adhesives, are suitable for the second adhesive layer. The suitable adhesives are described above. The second adhesive layer can be the same as the first adhesive layer or it can be different.

Also disclosed herein are electrodes prepared from the transfer tape articles described above. In some embodiments, the electrode comprises a substrate with a conductive surface, and a conductive transfer tape layer in contact with at least a portion of the conductive surface of the substrate. The conductive transfer tape layer is either the conductive transfer tape layer described above, or the multi-layer conductive transfer tape layer described above.

In some embodiments, the conductive transfer tape layer comprises a first layer of adhesive comprising a first major surface and a second major surface and a discontinuous layer of electrically conductive particles. A portion of the first major surface of the first adhesive layer, and at least some of the electrically conductive particles are in contact with the conductive surface of the substrate. The electrically conductive particles comprise shaped particles with at least one point. The first layer of adhesive envelopes the conductive particles, and at least one point of at least one of the electrically conductive particles protrudes from the second major surface of the conductive transfer tape layer.

In other embodiments, the conductive transfer tape layer is a multi-layer construction. In some embodiments, the multi-layer construction comprises a continuous or a discontinuous non-conductive support layer with a first major surface and a second major surface wherein the first major surface of the support layer is in contact with the second major surface of the first layer of adhesive, and a second adhesive layer with a first major surface and a second major surface, where the first major surface of the second adhesive layer is in contact with the second major surface of the support layer. In these embodiments, the support layer and the second adhesive layer also envelope the electrically conductive particles, and at least one point from at least one particle protrudes through the second major surface of the second adhesive layer.

A wide range of substrates are suitable in the electrodes of the current disclosure. The substrates are electrically conductive and include a sensor, monitor, or other electronic device, or can be attached and in electrical contact with a sensor, monitor, or other electronic device. Particularly suitable are wearable electronic medical devices.

Also disclosed are methods of preparing electrodes, where the method includes forming a conductive transfer tape article and using the conductive transfer tape article to form an electrode. In some embodiments, the method comprises providing a substrate with a conductive surface, wherein the substrate comprises an electronic device, providing a conductive transfer tape article with a first major surface and a second major surface with at least one release liner on the first major surface of the conductive transfer tape article, and removing the release liner from the conductive transfer tape article to expose the first major surface of the conductive transfer article, and contacting first major surface of the conductive transfer tape article to the conductive surface of the substrate.

The conductive transfer tape articles are described above. In some embodiments, the conductive transfer tape article comprises a release liner with a first major surface and a second major surface and a conductive transfer tape layer with a first major surface and a second major surface, where the first major surface of the conductive transfer tape layer is adjacent to the second major surface of the release liner. As described above, the conductive transfer tape layer comprises at least a first layer of adhesive comprising a first major surface and a second major surface, where a portion of first major surface of the first adhesive layer is in contact with the second major surface of the release liner, and a discontinuous layer of electrically conductive particles where at least some of the electrically conductive particles are in contact with the second major surface of the release liner. The electrically conductive particles comprise shaped particles with at least one point, the first layer of adhesive envelopes the conductive particles, and at least one point of at least one of the electrically conductive particles protrudes from the second major surface of the transfer tape layer. Thus, an electrical pathway thorough the conductive transfer tape layer via the electrically conductive particles is present.

In some embodiments, as described above, the conductive transfer tape layer is a multi-layer construction comprising a first adhesive layer, a support layer and a second adhesive layer. Each of these layers are described above.

In some embodiments, the prepared electrode further comprises a second release liner, where the second release liner is in contact with the protruding points of the electrically conductive particles and also is in contact with at least a portion of the second major surface of the conductive transfer tape layer. In embodiments where the conductive transfer tape layer comprises the first adhesive layer, the second release liner is in contact with the second major surface of the first adhesive layer. In embodiments where the conductive transfer tape layer is a multi-layer construction, the second release liner is in contact with the second adhesive layer. When the electrode is to be used, this second release liner can be removed to expose the protruding points of the electrically conductive particles which can be contacted to a skin substrate.

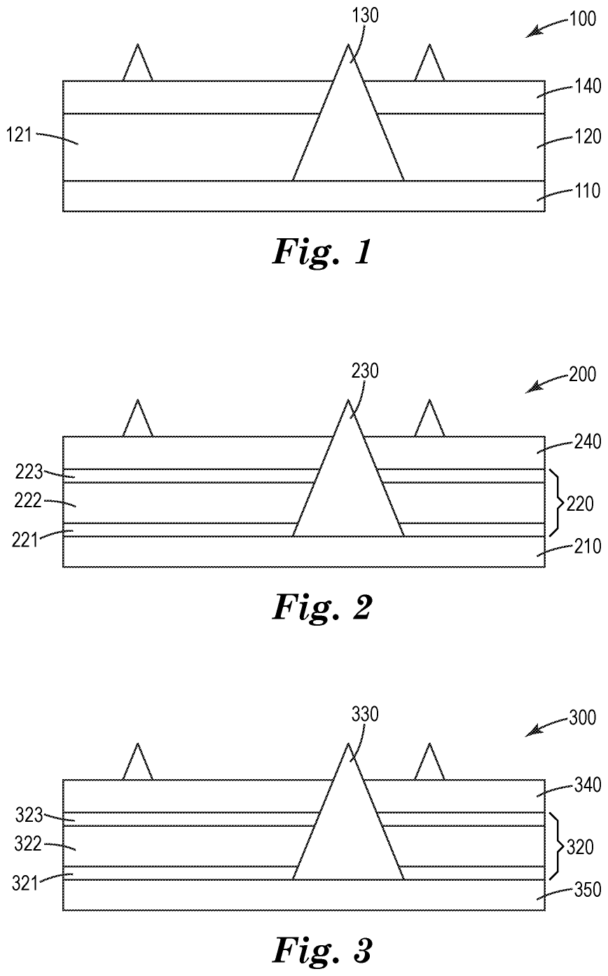

The transfer tape articles of this disclosure can be further understood by reference to the figures. FIG. 1 shows transfer tape article 100. Transfer tape article 100 comprises first release liner 110, electrically conductive transfer tape layer 120, electrically conductive shaped particles 130, and optional second release liner 140. Conductive transfer tape layer 120 is single layer of adhesive 121.

FIG. 2 shows transfer tape article 200. Transfer tape article 200 comprises first release liner 210, electrically conductive transfer tape layer 220, electrically conductive shaped particles 230, and optional second release liner 240. Conductive transfer tape layer 220 is multi-layer article comprising first adhesive layer 221, support layer 222, and second adhesive layer 223.

FIG. 3 shows a dry electrode article that can be prepared using the transfer tape articles of this disclosure, specifically a transfer tape article as in FIG. 2. Dry electrode article 300 comprises substrate 350, electrically conductive transfer tape layer 320, electrically conductive shaped particles 330, and optional second release liner 340. Conductive transfer tape layer 320 is multi-layer article comprising first adhesive layer 321, support layer 322, and second adhesive layer 323. The substrate 350 is electrically conductive and include a sensor, monitor, or other electronic device, or can be attached and in electrical contact with a sensor, monitor, or other electronic device. Particularly suitable are wearable electronic medical devices.

### Examples

These examples are merely for illustrative purposes only and are not meant to be limiting on the scope of the appended claims. All parts, percentages, ratios, etc. in the examples and the rest of the specification are by weight, unless noted otherwise. Solvents and other reagents used were obtained from Sigma-Aldrich Chemical Company, St. Louis, Mo., unless otherwise noted.

### Production of Precision Shaped Grain (PSG) Scalloped Tetrahedrons

The ceramic tetrahedrons were produced according to the procedures outlined in U.S. Patent Publication No. US 2016-0214903. Nano-zirconia shaped particles were made by casting a zirconia sol containing organic modifiers into molds and curing the sol to form gels that replicate the mold geometry and surface features. The gels were then further processed to form fully sintered particles that also replicated the mold geometry and surface features but were ˜55% smaller than the original mold dimensions. This process was used to create scalloped tetrahedra particles approximately 350 micrometers tall.

### Preparation of Silver Coated Zirconia Scalloped Tetrahedron Particles by Physical Vapor Deposition

The scalloped tetrahedrons were coated with silver using a physical vapor deposition method similar to that described in U.S. Provisional Patent Application No. U.S. 62/760,351.

### Assembly of Transfer Adhesive

Approximately 0.1 grams of silver-coated scalloped tetrahedral particles were sprinkled onto a 2″×2″ (5 centimeter×5 centimeter) square of silicone-coated paper adhesive release liner. A 17 gsm nylon nonwoven scrim (Cerex Advanced Fabric, Pensacola, FL) was laminated to 2.5 mil (64 micrometer) thick acrylate “stick to skin” pressure sensitive adhesive (prepared as described in Ulrich RE 24,906) on a silicone coated paper adhesive release liner. This construction was placed over the top of the scattered particles with the nonwoven layer facing the particles. The adhesive, nonwoven, and particle construction sandwiched between 2 silicone coated paper adhesive release liners was laminated with a Western Magnum XRL 120A laminator (El Segundo, CA) at 110° C., 80 PSI and 1.5 ft/min (45 cm/min). The conditions used for lamination caused some of the adhesive to migrate through the nonwoven so that there is adhesive exposed on both sides of the nonwoven to form a construction such as is shown in FIG. 2. The tetrahedron tips protruded through the surface of one side of the adhesive and the bases of the tetrahedrons were in the plane of the second adhesive surface.

### Applying Transfer Adhesive to Conductive Substrates

A release liner (designated release liner 1, 210, in FIG. 2) was removed from the transfer tape article. The exposed surface containing the adhesive surface and tetrahedron bases was placed on 2 different conductive substrates. The first substrate was the backing of 3M Red Dot Electrode 9650. This backing is polyester film coated with a continuous layer of conductive carbon (the printed silver/silver chloride stripes and hydrogel adhesive that are normally part of the 9650 construction were not included). The second substrate was a steel panel, 2″×5″ (5 centimeter×13 centimeter), #302 or 304 ANSI Stainless Steel. Both constructions were laminated with a Western Magnum)aL, 120A laminator at 110° C., 80 PSI (0.55 MPa) and 1.5 ft/min (45 cm/min) to ensure adequate adhesion of the PSA and contact of the tetrahedron bases to the conductive substrate.

Additional samples were constructed by laminating a comparative construction similar to the FIG. 2 adhesive construction to the same two conductive substrates. This comparative construction was identical to the FIG. 2 adhesive construction, except that it did not contain the electrically conductive tetrahedrons.

### Electrode Impedance Testing

The second release liner (designated release liner 2, 240, in FIG. 2) was removed to expose the PSA surface with protruding tetrahedron points. The surface of the adhesive and the tetrahedron points were covered with hydrogel adhesive squares taken from 3M 2360 resting electrodes. One 3M 2360 electrode was placed in contact with the hydrogels. An additional sample was constructed for comparison by placing two 3M 2360 electrodes back to back with their hydrogel surfaces in contact. The electrode assemblies were connected to an ET-3P ECG/Defibrillator Electrodes Tester from Xtratek (Lenexa, Kans.). The impedance of the back to back electrode set up was tested at a frequency of 10 Hz, and the results are provided in Table 1. Per AAMI EC12:2000 standard for disposable ECG electrodes, no single pair of electrodes may exceed 3 kΩ impedance. The dry electrode constructions with penetrating tetrahedron particles met this criterion, while those without the tetrahedron particles did not. This confirms that the tetrahedron bases are in sufficient contact with the conductive substrate and the tetrahedron tips are protruding from the adhesive surface.

TABLE 1
Electrical impedance test results.

| Electrode 1 | Electrode 2 | Impedance (Ω) @ 10 Hz |
|---|---|---|
| 3M2360 | Dry electrode with 3M 9650 backing and | 2071 |
| | tetrahedron particles | |
| 3M2360 | Dry electrode with 3M 9650 backing (no particles) | >3000 (signal not |
| | | detectable) |
| 3M2360 | Dry electrode with stainless steel backing and | 2460 |
| | tetrahedron particles | |
| 3M2360 | Dry electrode with stainless steel backing (no | >3000 (signal not |
| | particles) | detectable) |
| 3M2360 | 3M2360 | 229 |

### On-Skin Impedance Testing

Electrodes were placed approximately 4 inches (10.2 centimeters) apart on a volunteer's arm and pressed in place with moderate finger pressure for roughly one second. Impedance between the electrodes was measured immediately after application using an EIM-105-10 Hz Prep-Check Electrode Impedance Meter (General Devices, Inc., Indianapolis, Ind.). Fresh sites were used for the application of each electrode. For each pair, the distance between centers was approximately 4 inches (10.2 cm). Table 2 presents the results from a series of pairs.

TABLE 2
On-skin impedance test results.

| Electrode 1 | Electrode 2 | Impedance (kΩ) @ 10 Hz |
|---|---|---|
| 3M2360 | Dry electrode with 3M 9650 backing and tetrahedron | 88 |
| | particles | |
| 3M2360 | Dry electrode with 3M 9650 backing (no particles) | >200 (signal not |
| | | detectable) |
| 3M2360 | Dry electrode with stainless steel backing and tetrahedron | 161 |
| | particles | |
| 3M2360 | Dry electrode with stainless steel backing (no particles) | >200 (signal not |
| | | detectable) |
| 3M2360 | 3M2360 | 191 |

What is claimed is:

1. A transfer tape article comprising:
a release liner with a first major surface and a second major surface; and
a conductive transfer tape layer with a first major surface and a second major surface,
wherein the first major surface of the conductive transfer tape layer is adjacent to the second major surface of the release liner, the conductive transfer tape layer comprising:
at least a first layer of adhesive comprising a first major surface and a second major
surface, wherein a portion of first major surface of the first adhesive layer is in contact
with the second major surface of the release liner;
a discontinuous layer of electrically conductive particles wherein at least some of the
electrically conductive particles are in contact with the second major surface of the
release liner, and wherein the electrically conductive particles comprise
shaped particles with at least one point, wherein the first layer of adhesive envelopes
the conductive particles, and wherein at least one point of at least one of the
electrically conductive particles protrudes from the second major surface of the
conductive transfer tape layer.

2. The transfer tape article of claim 1, wherein the conductive transfer tape layer is a multi-layer construction comprising:
a continuous or a discontinuous conductive or non-conductive support layer with a
first major surface and a second major surface wherein the first major surface of the
support layer is in contact with the second major surface of the first layer of adhesive,
wherein the support layer envelopes the conductive particles; and
a second adhesive layer with a first major surface and a second major surface, wherein
the first major surface of the second adhesive layer is in contact with the second major
surface of the support layer.

3. The transfer tape article of claim 2, further comprising a second release liner with a first major surface and a second major surface, wherein the first major surface of the second release liner is in contact with at least one electrically conductive particle protruding from the second major surface of the transfer tape layer and is also in contact with at least a portion of the second major surface of the transfer tape layer.

4. The transfer tape article of claim 2, wherein the support layer comprises an essentially continuous layer, comprising a film, a web, a sheet, or a foam.

5. The transfer tape article of claim 2, wherein the first adhesive layer and the second adhesive layer both comprise a pressure sensitive adhesive.

6. The transfer tape article of claim 1, wherein the electrically conductive particles comprise non-compressive shaped particles with a conductive metal coating wherein at least one dimension of the shaped particles is 175-1,500 micrometers.

7. The transfer tape article of claim 6, wherein the first adhesive layer has a thickness that is 25-250 micrometers less than the at least one dimension of the shaped particles.

8. The transfer tape article of claim 2, wherein the electrically conductive particles comprise non-compressive shaped particles with a conductive metal coating wherein at least one dimension of the shaped particles is 175-1,500 micrometers.

9. The transfer tape article of claim 8 wherein the conductive transfer tape layer has a thickness that is 25-250 micrometers less than the at least one dimension of the shaped particles.

10. The transfer tape article of claim 6, wherein the non-compressive shaped particles comprise ceramic shaped particles.

11. An electrode comprising:
a substrate with a conductive surface; and
a conductive transfer tape layer in contact with at least a portion of the conductive surface of the substrate, the conductive transfer tape layer comprising:
at least a first layer of adhesive comprising a first major surface and a second major
surface, wherein a portion of first major surface of the first adhesive layer is in contact
with the conductive surface of the substrate;
a discontinuous layer of electrically conductive particles wherein at least some of the
electrically conductive particles are in contact with the conductive surface of the substrate, and wherein the electrically conductive particles comprise
shaped particles with at least one point, wherein the first layer of adhesive envelopes
the conductive particles, and wherein at least one point of at least one of the electrically conductive particles protrudes from the second major surface of the conductive transfer tape layer.

12. The electrode of claim 11, wherein the substrate comprises an electronic device.

13. The electrode of claim 11, wherein the conductive transfer tape layer is a multi-layer construction comprising:
a continuous or a discontinuous conductive or non-conductive support layer with a first major surface and a second major surface wherein the first major surface of the
support layer is in contact with the second major surface of the first layer of adhesive,
wherein the support layer envelopes the conductive particles; and
a second adhesive layer with a first major surface and a second major surface, wherein
the first major surface of the second adhesive layer is in contact with the second major
surface of the support layer.

14. The electrode of claim 13, wherein the support layer comprises an essentially continuous layer, comprising a film, a web, a sheet, or a foam.

15. The electrode of claim 13, wherein the first adhesive layer and the second adhesive layer both comprise a pressure sensitive adhesive.

16. The electrode of claim 11, wherein the first adhesive layer has a thickness that is 25-250 micrometers less than the at least one dimension of the shaped particles.

17. The electrode of claim 13, wherein the conductive transfer tape layer has a thickness that is 25-250 micrometers less than the at least one dimension of the shaped particles.

18. The electrode of claim 16, wherein the non-compressive shaped particles comprise ceramic shaped particles.

19. A method of preparing an electrode comprising:
providing a substrate with a conductive surface, wherein the substrate comprises an electronic device;
providing a conductive transfer tape article with a first major surface and a second major surface wherein the conductive transfer tape article comprises:
a release liner with a first major surface and a second major surface; and
a conductive transfer tape layer with a first major surface and a second major surface,
wherein the first major surface of conductive transfer tape layer is adjacent to the second major surface of the release liner, the conductive transfer tape layer comprising:
at least a first layer of adhesive comprising a first major surface and a second
major surface, wherein a portion of first major surface of the first adhesive layer is in contact with the second major surface of the release liner;
a discontinuous layer of electrically conductive particles wherein at least some
of the electrically conductive particles are in contact with the second major surface of the release liner, and wherein the electrically conductive particles comprise shaped particles with at least one point, wherein the at least first layer
of adhesive envelopes the conductive particles, and wherein at least one point
of at least one of the electrically conductive particles protrudes from the second
major surface of the transfer tape layer;
removing the release liner from the conductive transfer tape article to expose the first major surface of the conductive transfer tape layer; and
contacting first major surface of the conductive transfer tape article to the conductive surface of the substrate.

20. The method of claim 19, wherein the conductive transfer tape layer further comprises a support layer with a first major surface and a second major surface, wherein the first major surface of the support layer is in contact with the second major surface of the first adhesive layer, and a second adhesive layer with a first major surface and a second major surface, wherein the first major surface of the second adhesive layer is in contact with the second major surface of the support layer, and wherein the conductive transfer tape layer has a thickness that is 25-250 micrometers less than the at least one dimension of the shaped particles.
